(19) United States
(12) Patent Application Publication
(10) Pub. No.: US 2013/0226183 A1
(43) Pub. Date: Aug. 29, 2013

(54) SURGICAL KIT AND A FACILITATED PROTOCOL FOR THE IMPLANTATION OF ACETABULAR PROSTHESIS

(71) Applicants: Ping Xie, Ashland, MA (US); Xue Li, Ashland, MA (US)

(72) Inventors: Ping Xie, Ashland, MA (US); Xue Li, Ashland, MA (US)

(21) Appl. No.: 13/770,269

(22) Filed: Feb. 19, 2013

Related U.S. Application Data
(60) Provisional application No. 61/603,751, filed on Feb. 27, 2012.

(51) Int. Cl. A61B 17/16; A61B 19/02; A61F 2/00

(52) U.S. Cl. CPC A61B 17/1666; A61F 2/0095; A61B 19/026
USPC 606/80; 206/570

(57) ABSTRACT

A ready-for-use surgical kit and a facilitated protocol for the implantation of acetabular prosthesis in hip replacement surgery have been introduced. In particularly, a ready-for-use package includes both a set of the artificial acetabular prosthesis and a set of specific reamer(s) with a predefined spherical cutting profile. According to a protocol, surgeon could accurately select a package including both a size of acetabular prosthesis and a corresponding finishing reamer according to a pre-measurement and bone condition of a respective patient, and then patient-specifically remedy a defected acetabulum into a full hemispherical form having a personalized dimension by the reamer(s) within the package during operation.

### CROSS-REFERENCE TO RELATED APPLICATIONS

The present invention claims the benefit of U.S. Provisional Patent Application 61/603,751, filed Feb. 27, 2012, which is hereby incorporated by reference herein.

### FIELD OF THE INVENTION

The present invention relates to surgical kit(s) and a facilitated protocol(s) for the implantation of acetabular prosthesis. In particular, the invention relates to how to accurately remedy the acetabulum by tool(s) individually selected in order to precisely press fit the acetabular prosthesis therein in the event of the surgery of the hip joint for use in either a partial or total hip replacement procedure.

### DESCRIPTION OF THE PRIOR ART

A process of reaming the defected acetabulum is definitely complicated and should always be considered critical to the overall success of the implantation for many reasons. Acetabular reamers in the prior art generally include a series set of reusable, hemispherical dome-like reamer with a progressive increment on diameter. An interval of the diameter between adjacent reamer is about 2 mm. After decades of practice, from the point of view of clinic, there are at least few concerns associated with using this kind of reusable reamer set in clinic.

One concern is: a size and a spherical geometry of the acetabulum reamed should ideally be identical with the dimension of the reusable reamer used. But in fact, they are quite different due to a well-known, fatal weakness on the cutting principle it followed.

Further concern is: the general procedure of the hip joint replacement instructs that the dimension difference between the artificial acetebular cup intended to be used in the press-fit process and the acetabulum reamed is about 2 mm and remains same for all patient, regardless their wide spread variation on the bone quality and capability to receive the cup. It is obviously that various final size of the acetabulum reamed should be desirably needed from case to case, according to the information and diagnosis based upon each individual patient's bone elasticity. A preferred solution for such a surgery could be a tooling set, which allows that a surgeon is able to elect: what is a right size of the acetabular reamer for using and what is a customized protocol for correctly doing it, which includes how to choose right tools. If do so, it might be called a semi-customized design of surgery. Unfortunately, the current available tools did neither have such a function nor were able to do so.

Furthermore concern: it is also well known, the accurate final size and spherical geometry of the acetabulum reamed is so critical to the outcome of the press-fitting process. In the reality, the random and repeat usages of the reusable reamer cause that its sharpness of the cutting teeth and accurate dimension of the hemispherical shell of the reamer are slightly varied away from original one. The degree of such as abrasion is so arbitrary and undetectable from piece to piece within same set of reamer, and even though on the same dome reamer, it could also be varied from area to area. The outcomes of cutting quality from such a dull reamer, particular one used in the last step of reaming process, would be unknown or imaginable in term of both the size and the spherical geometry of the acetabulum. If either one above is the case, the efficacy of the press-fit is definitely suspicious regarding to the stability and functions of the prosthesis implanted in term of both the short and long term concerns. In order to overcome such problems described above, to use a reamer always having fresh and sharp cutting edge/teeth as a final reamer should be considerable. So the disposable/low cost reamer becomes one of ideal candidates.

An additional concern: by the same token, a set of reusable acetabular reamer commercially used for cutting bone now are often difficult to remove and to clean the residues, such as prions, soft tissue and others contaminated on the surface of the tool from some prior to use. It becomes most concerns to make sure that such a manner does not cause a cross contamination occurred between uses, particularly in cases of a patient with infection disease. It is definitely desirable on utilizing a low cost cutting tool that is intended for a single or few use(s). If does so, the cutting tool could be simply discarded after using.

The last concern: due to aforementioned problems or unmet needs above, there are various ideas, which have claimed patent rights for a disposable or low cost reamer of cutting defected acetabulum described in the prior art. Majority of them claimed have focused upon how to simplify a structure and manufacturing protocol of the reamer to make it at a cost as low as acceptable. Actually, the latent problem faced for the disposable reamer is that the higher total cost of using the reamer set in clinic is far beyond the issue of lowering the cost of making a reamer itself. Therefore, if one just follows inertia thought of what the reusable reamer did and goes through a regular procedure to estimate the total cost of using disposable reamer, one will finally end up a conclusion of that there is no commercial future or potential for using disposable reamer or likes in the real market at all, because there are a significant cost and overhead due to its post treatment, such as its packaging, sterilization and handling. Apparently it should consider all cost affiliated with its hardware/infrastructure invested, manufacturing, cleaning, packaging, sterilizing and discharging it. So how to reduce the total cost challenges the future of the disposable reamer.

What is needed therefore are surgical kits, which can be used in a surgery of a specific patient to efficiently and economically implant the acetabular cup.

Still further, what is needed therefore are protocols of using such a surgical kit, for instance, such a disposable reamer(s), by which surgeon is able to customize or design an individual procedure for each patient in order to provide an accurate hemispherical form in size and quality and thus ensures precise and efficient implantation of acetabular prosthesis in a convenient and a cost effective manner.

### SUMMARY OF THE INVENTION

The present invention relates to a unique surgical kit and a facilitated protocol for a surgery of implanting acetabular prosthesis. The three types of surgical kit(s) disclosed herein, no intent to limit the invention to the particular form disclosed, could be a good option of adapting into individual surgeon's experience, taste or skills for fitting into a particular patient's condition or profile. The preferred protocol facilitated with a corresponding surgical kit of use is also disclosed herein, but individual surgeons may still elect to use them in the preferred fashions.

As such, a first object is that the surgical kit and protocol described in the present invention would allow that the surgeon is able to efficiently use predefined surgical Parameters from measurement of patient prior to a surgery, then determine/elect a specific prosthesis and tooling set, as well as select a customized protocol for each individual surgery. It includes a key step of selecting a low cost finishing reamer from a few of options of its dimension, which provides advantage of being able to deal with the situation of patient having a specific bone condition.

A further object is that the surgical kit and protocol described in present invention provide solutions to overcome concerns on the uncertain quality of the acetabulum reamed during the final reaming step because of unknown sharpness of the conventional reamer set. Contrarily, to use a disposable or low cost reamer with a fresh sharpness as a final reamer would firmly ensure that the dimension and quality of the cavity reamed meet the qualities for the prefect press-fitting.

It also is a further object that the surgical kit and protocols described in the present invention, to be more specifically, that both acetabular prosthesis and at least one low cost (fully disposable or partially disposable) finishing reamer packaged together within a same surgical kit(s), brings about a realistic and affordable solution toward market acceptances of using a low cost reamer in clinic, based upon the cost effective manner applied.

Moreover, the present invention is designed to overcome at least one of the aforementioned problems or unmet needs realized in clinic.

### BRIEF DESCRIPTION OF THE DRAWINGS

FIG. 1 shows a popular arrangement of commercially packaging the components of the acetabular prosthesis assembly for using on the current clinic.

FIG. 2 shows a typical arrangement of components packaged within a surgical kit described in the present invention for implantation of acetabular prosthesis, which generally comprises at least one prosthesis compartment and one tooling compartment, respectively.

FIG. 3 shows a preferred arrangement of components packaged within a surgical kit, described in details in the first preferred embodiment regarding to the surgical kit, type 1.

FIG. 4 shows other preferred arrangement of components packaged within a surgical kit, described in the second preferred embodiment regarding to the surgical kit, type 2.

FIG. 5 shows another preferred arrangement of components packaged within a surgical kit, described in the third preferred embodiment regarding to the surgical kit, type 3.

FIG. 6 shows a block diagram of a conventional protocol used in the current clinic to implant the acetabular prosthesis, which is usually fulfilled by dome-like, reusable reamers.

FIG. 7 shows a block diagram of a general protocol to implant the acetabular prosthesis by using the surgical kit(s) described in the present invention.

FIG. 8 shows a block diagram of the preferred implantation protocol 1 corresponding to using the surgical kit, type 1.

FIG. 9 shows a block diagram of the preferred implantation protocol 2 corresponding to the surgical kit, type 2.

FIG. 10 shows a block diagram of the preferred implantation protocol 3 corresponding to the surgical kit, type 3.

### SPECIFIC DESCRIPTION OF THE PREFERRED EMBODIMENT

While the invention herein is susceptible to various modifications and alternative forms, each specific embodiment thereof has been shown by way of example in the drawings and will herein be described in detail. It should be understood, however, that there is no intent to limit the invention to the particular form disclosed, but in the contrary, the intention is to cover all modifications, equivalents, and alternatives falling within the spirit and scope of the invention as defined by the appended claims.

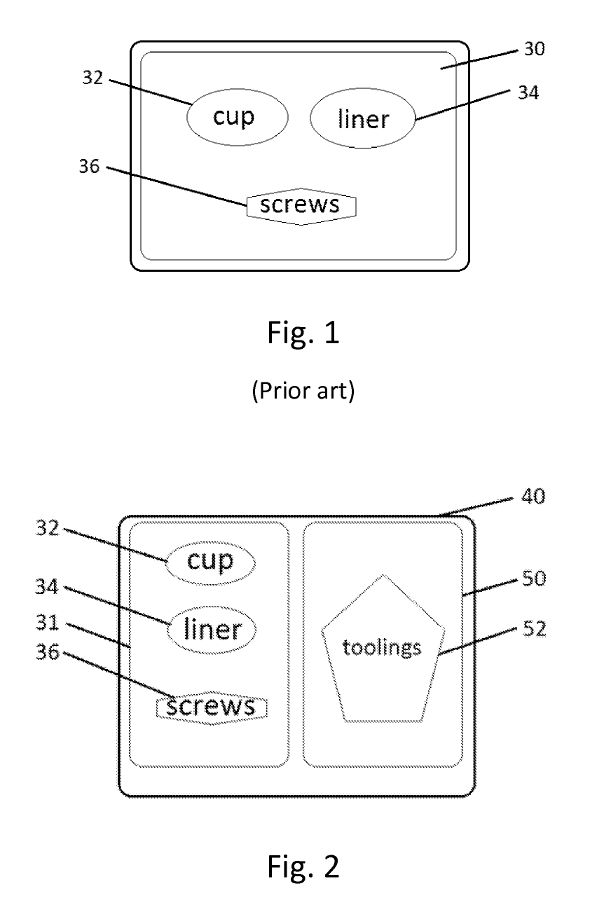

Referring now to FIG. 1, there is shown a conventional arrangement 30 of commercially packaging the components of artificial acetabular prosthesis in the prior art. Normally, the acetabular prosthesis package herein comprises at least a few of or three sub-compartments suitable for packaging one acetabular cup 32, one corresponding liner 34 and the fixation screws 36, respectively. The typical label outside the package/box indicates at least the size of the acetabular prosthesis included. Each component included within the package has properly been cleaned, wrapped/packaged and sterilized in a standard manner. For instance, sterilization can be performed using any suitable technique, for example radiation, such as gamma radiation. During the operation, the surgery assistant only needs to open the specific package selected and follow the protocol to step by step hand over each component to surgeon in the surgery sequences without any additional treatment on such the component.

As known, in a conventional surgery of the hip replacement, such packages delivered to the operation room by the device manufacturer/company should include all set (in size) of the acetabular prosthesis, because, at the time prior to implantation, there is no information available regarding to characteristics of each individual patient and what is the size of the prosthesis intended to be used.

FIG. 2 has shown a typical, in general, arrangement of a surgical kit(s) 40 described in the present invention, which comprises at least two compartments, an acetabular prosthesis compartment 31 and a tooling compartment 50, respectively. To be noticed, all surgical kits or packages described herein are a typical terminally sterilized, aseptic package including either medical device or tools, or both. They are clean and ready for surgical use.

Typically, the arrangement within the acetabular prosthesis compartment 31 comprises at least a few of or three sub-compartments suitable for packaging one acetabular cup 32, one corresponding liner 34 and a few of fixation screws 36, respectively, with the specific size that conformed to the label marked outside the package and in the similar manner as one in commercial package 30 as mentioned above.

To be noticed, the definition of “a single use” used/cited herein and thereafter could mean “either fully disposable or partially disposable or both”.

Within a tooling compartment 50 herein, there is at least one or a few of sub-compartment, in most preferred matter, for packaging either one low cost cutting tool or a set of low cost cutting tools, respectively. Obviously, in most preferred matter, the low cost cutting tool packaged with acetabular prosthesis assembly together is herein indicated that it could be of a single use with an extended meaning of either fully disposable or partially disposable or both, but under certain circumstances it might be or is allowable to be reusable by a repackaged manner.

It should be emphasized herein, that the definition of “low cost” used/cited within this present invention should include implication of that either disposable or partially disposable tool or both is a low cost tool for a single use; or reusable tools end up at a low cost for each individual single use after its multiple uses”. In order word, if, in some circumstance, each actual average using cost of reusable tool(s) might or should be low as or lower than a disposable one, the tool would/should also be a good candidate for this application. A precondition to do so is, for example, that the reusable tool herein should always be fresh and sharp and able to accurately and efficiently ream all area of hemispherical acetabulum after post maintenances. The post maintenances after each use should at least include cleaning, re-sharping the cutting edge/teeth, checking the dimension of the tool and repackaging. In briefly, no matter the tool is disposable or not, if any tools can fit into the following criteria, it could be used and conform to the idea of the present invention. The criteria are: the tool possesses:

1) a low total cost for each single use,

2) a fresh, sharp cutting edges/teeth at each use, and

3) an accurate dimension of the tool at each use.

A dimension of the cutting tools herein is corresponding to and compatible with one of the acetabular prosthesis packaged with. Beyond the safety and cost issue, the function of the low cost cutting tool(s) herein is able to guarantee that the dimension and quality of acetabulum reamed would perfectly match the criteria of precisely installing an acetabular cup required by press-fit process, which might include 1) how tightly the acetabulum reamed can hold the acetabular cup and 2) how much the contact area of the surface can be matched each other between two items.

A first preferred embodiment regarding to a surgical kit, type 1:

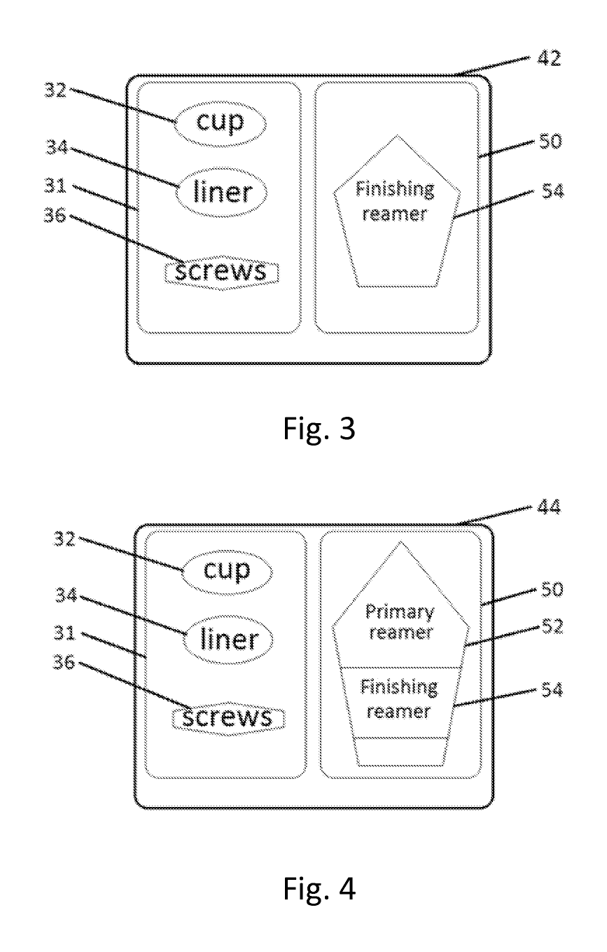

Furthermore, within a reference to FIG. 3, a surgical kit, type 1, 42, described in the present invention comprises two compartments, an acetabular prosthesis compartment 31 and a tooling compartment 50, respectively. Within the acetabular prosthesis compartment, each sub-compartment correspondingly packages one acetabular cup 32, one liner 34 that fits within the cup or a few of fixation screws 36, respectively. The specific size of acetabular cup 32 herein conforms to the label marked outside the package, as mentioned above. Within the tooling compartment 50, its sub-compartment has at least comprised, in most preferred matter, one low cost, finishing reamer 54 for finely cutting, which has a specific dimension coincided with the label marked outside the package. The finishing reamer 54 herein could be any type of a low cost reamer, regardless the cutting principle followed and the structure designed. For example, the low cost herein means a total cost for each single use, which could correspond to a manner of, but no intent to be limited, either fully disposable or partially disposable reamer, or repeatedly using a reusable reamer.

The finishing reamer 54 packaged herein should at least possess or satisfy the following characteristics and functions:

1. In the contrary to a conventional reaming process of the acetabulum, the finishing reamer 54 has always a fresh, sharp cutting edge/teeth and accurate, but predefined dimension, in order to finely remedy the acetabulum targeted and obtain high quality of acetabulum cavity in term of its dimension (accurate size and spherical geometry) and smooth surface.

2. To optimize the results of the press-fit procedure, there should be a few of the finishing reamer with varied predefined size available, corresponding to each specific dimension of acetabular cup intended to use, which allows surgeon to make a customized selection on reaming a final cavity size, based upon the bony characteristic information from each individual patient.

3. There is a universal driver interface associated with the structure of the finishing reamer 54 in order that the finishing reamer 54 is able to couple with and be driven by a same driving power source used in conventional reamer system, as dome-like reamer does.

4. As similar as a conventional reaming system, the finishing reamer can be operated either manually by surgeon or automatically by surgical robot.

As well known in clinics, the individual bone quality and bony elasticity are varied from patient to patient, which is a very important factor on affecting the performance of installing acetabular cup by press-fit method. In order to have an optimized performance of and ideal outcomes from press-fitting, the bone quality, particular its elasticity should be considered within the procedure selection of reaming. For instance, in case of younger patient with no disease history, the acetabulum bone is able to tolerate more elastic deformation, when press-fit performed, so the final size of the acetabulum reamed could be at least 2 mm smaller than one of the acetabular cup. In an opposite case of old age patient or patient with a disease, a mutation of the bone quality leads nowhere to known his/her bone elastic tolerance. So their best fitting size of acetabular cavity could be arbitrary. In clinical convenience, surgeon needs flexibility on such a fine judgment on the final cavity size.

Regarding to the selection on the dimension of the finishing reamer, since the standard size (diameter) of the reamer is increased by 2 mm interval, a size of the fine, finishing reamer should be able to fit into that 2 mm gap with a further fine interval, such as each 1 mm or 0.5 mm interval. A predefined size of a specific finishing reamer packaged with the corresponding acetabular prosthesis becomes a secondary characteristic of the surgical kit described in the present invention. Information about both acetabular prosthesis and the finishing reamer 54 herein should be identified on the label on the outside of the package box, when they are packaged. For example, 52 mm acetabular prosthesis assembly can be packaged with various size of finishing reamer: such as 50.5; 51.0; 51.5 or 52.0 mm diameter of reamer, respectively. The label on the outside of the box could be corresponding to 52A; 52B; 52C or 52D, or likes, respectively.

A second preferred embodiment regarding to a surgical kit, type 2:

Furthermore, with further reference to FIG. 4, a surgical kit, type 2, 44, described in the present invention still comprises two compartments, an acetabular prosthesis compartment 31 and a tooling compartment 50, respectively. The components packaged within the acetabular prosthesis compartment 31 remained same manners as one described in the first embodiment above.

Within a tooling compartment, there are, in most preferred matter, one low cost, primary reamer 52 for coarsely cutting and one low cost, finishing reamer 54 for finely cutting. Both reamers have their own predefined dimension. Obviously, within the same package set, the size of the primary reamer 52 is smaller than one of the finishing reamer 54. They could be packaged either within same sub-compartment or separately. The sizes of both reamers coincide with the label marked outside the package. Both reamers could be any type of a low cost reamer, regardless the cutting principle followed, the structure designed and the manner used. For example, the low cost herein means a total cost for each single use, which could correspond to a manner of, but no intent to be limited, either fully disposable or partially disposable reamer, or repeatedly using a reusable reamer at a lower running cost.

The primary reamer 52 packaged herein should at least possess or satisfy the following characteristics and functions:

1. With a respect of its structure and sharp cutting edge/teeth, it is able to rapidly sweep out all cartilage and a minimum layer of cancellous bone within the acetabulum after a few of rotation, more particularly, any tissue localized within area around the apex area of hemisphere.

2. No matter the initial dimension of the acetabulum, the primary ream 52 is able to cover all range up to the size of 2 mm less than one of the acetabular cup 32 intended to be used.

3. There is a feature affiliated with the primary reamer 52, by which the reamer has a function to pick up the debris generated during cutting.

4. The operation could be fulfilled either manually by surgeon or automatically surgical robot.

5. A driver interface affiliated with the structure/frame of the primary reamer 52 could be selected from groups either to couple with a standard universal shaft used by conventional system or likes, or one having own special design.

The finishing reamer 54 herein packaged should possess and satisfy the same characteristics and functions as some described in the first preferred embodiment above, but it should possesses a same driver interface as one of the primary reamer 52 within same package.

A third preferred embodiment of a surgical kit, type 3:

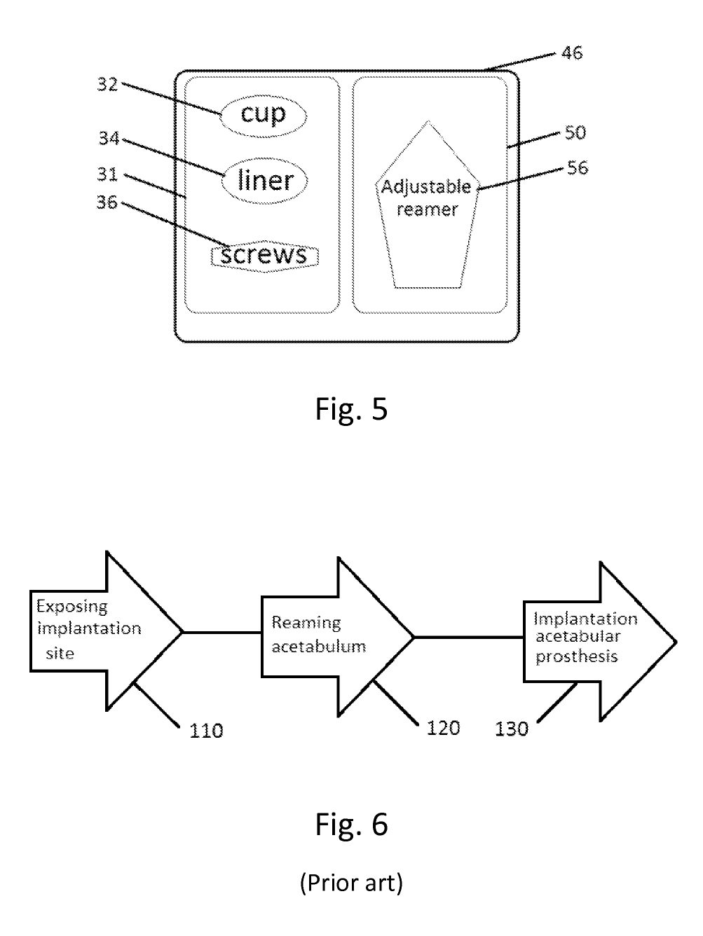

Furthermore, with further reference to FIG. 5, a surgical kit, type 3, 46, described in the present invention should still comprise two compartments, an acetabular prosthesis compartment 31 and a tooling compartment 50, respectively. The components within the acetabular prosthesis compartment 31 remained same as one described in the first embodiment.

Within the tooling compartment 50, there is, in most preferred matter, at least one low cost, expandable/adjustable reamer 56 with an accurate, but predefined, final dimension intended to use. The size or adjustable range of the adjustable reamer coincides with the label marked outside the package. The adjustable reamer could be any type adjustable reamer designed and manufactured at a low cost, regardless cutting and expanding principle it followed and the structure adapted. For example, the low cost herein means a total cost for each single use, which could correspond to a manner of, but no intent to be limited, either fully disposable or partially disposable reamer, or repeatedly using a reusable reamer at lower running cost.

The adjustable reamer 56 packaged herein should possess or satisfy the following characteristics and function:

1. With a respect of its rational structure and fresh, sharp cutting edge/teeth, the adjustable reamer 56 herein is able to rapidly, but moderately, sweep out all cartilage and a minimum layer of cancellous bone within the acetabulum after a few of rotations, and then conveniently, smoothly, step by step expand its cutting profile up to the dimension intended to be. In other word, no matter what is the dimension of original acetabulum premeasured, the reamer is able to cover all range and quality targeted in term of the size, spherical geometry and a specific area, such as, particularly, any tissue localized within area around apex area of hemisphere.

2. Expanding reamer's dimension could be conveniently managed either in an intermittent or continual manner.

3. Its operation could be performed either manually by surgeon or automatically by a surgical robot.

4. There is a unique driver interface affiliated with the adjustable reamer for coupling with the shaft of the driving mechanism. The shaft has functions of both an axle rotation and regulating the cutting profile of the reamer.

More specific, the adjustable reamer 56 is a reamer which is able to initially start a reaming at any dimension or status of original acetabulum and accurately, efficiently, step by step ream up to the size predefined. In comparison to two prior surgical kits 42 and 44 described above, the one reamer herein could perform similar as what is done by others. It saves a significant time, cost and avoids unnecessary damages on neighbor tissue during exchanging the reamer head and repeat entrances.

Once again, it should be understood, however, that there is no intent to limit the invention to the particular form disclosed, for instance, all types of low cost tools packaged with acetabular prosthesis assembly together as discussed in all embodiments above could be intended for a single use, but under certain circumstances they might be or are allowable to be reusable in a manner of that the durable reamer has been repackaged after a post treatment. Also the single use tool above is indicated to be either fully disposable, partially disposable or others of running at lower cost.

In order to well perform the surgical kit(s) discussed in preferred embodiments of the surgical kits above into clinics, the preferred protocols of corresponding surgical kit in hip replacement surgery will now be set forth. Each preferred embodiment of the protocol discussed as following, but no limit within such embodiments and away from a scope and sprite of the present invention, is corresponding to each preferred embodiment of the surgical kit(s) in previous discussion.

With further reference to FIG. 6, a block diagram of the conventional protocol in the prior art for implanting an acetabular prosthesis cup during a hip joint replacement surgery in current clinic has, in briefly, comprises steps: 1) pre-treatment of the surgery 110, includes such as adequate exposure of the joint for surgery and cutting the femoral head and neck to expose the acetabulum site. 2) The acetabulum is prepared 120 by using a set of hemispherical reamer to remove cartilage down to the bleeding bone. The typical tools used in this step are a set of acetabular reamers, which are hemispherical in shape and have sharp raised portions, such as that of a cheese grater. 3) Selecting a size of acetabular prosthesis according to the size of the last reamer head used and following a standard protocol of implantation of acetabular prosthesis 130. For instance, once the acetabulum surface is prepared, the acetabular component is implanted, either by cementing in place or by press fitting a metal shaft shell for bony in-growth.

In this kind of operation, it happened that there is variability or uncertain on selection of the size of acetabular cup, even though for a same patient, but performed by different surgeon. It might be due to the experience, skill of surgeon and some human factors.

Preferred protocol of Use:

A general protocol:

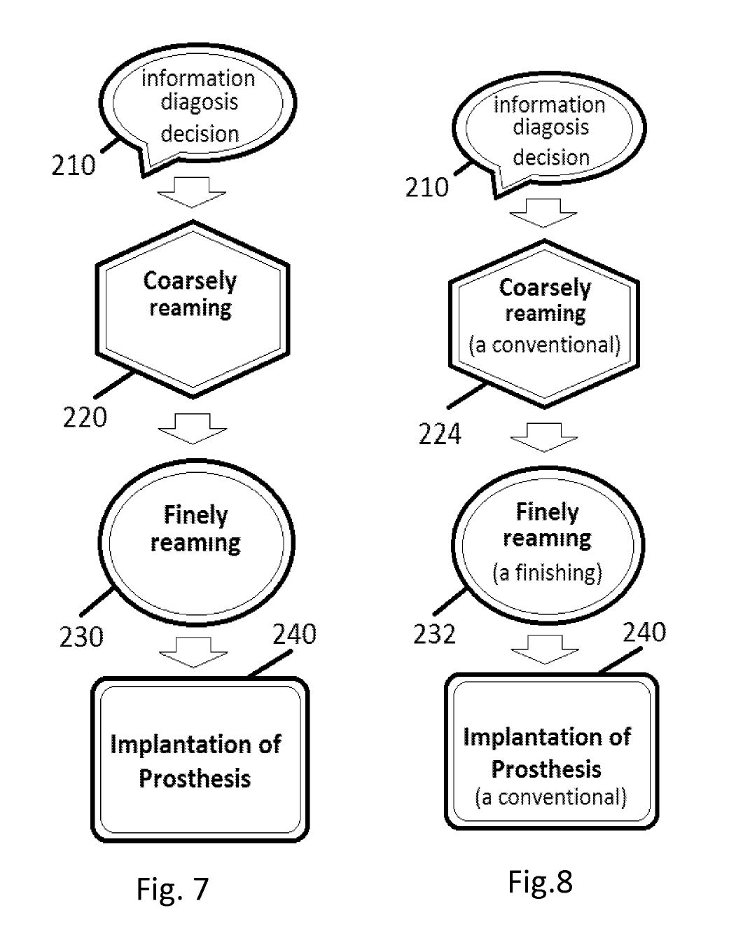

With further reference to FIG. 7, in the contrary to a conventional protocol above, a block diagram has shown a general protocol related to the present invention for customized installing an acetabular prosthesis during a hip joint replacement surgery. The general protocol comprises the following procedures as shown in processing sequences:

1. A Procedure of information collection 210:

This procedure, generally, includes steps of information collection of patient, data analysis and decision making by surgeon, respectively. Measuring one or more dimensions of the intended implantation site or the dimensions of the area surrounding the intended implantation site now becomes a standard procedure in many type of customized surgery. Herein we emphasize only acetabulum portion, but also the measurement simultaneously carried includes other area of defected joint, such as proximal section of the femur for replacement surgery on femoral side. Information collection from patient herein is useful for identifying the condition of the defected joint and determining the surgical parameters, such as the size of acetabular cavity defected and characteristics related to an accurate implantation of the acetabular prosthesis. In the contrary to conventional protocol of the prior art discussed above, information collection from each individual patient as well as accurate diagnosis are a very important step in decision making by surgeon and should at least includes both the characteristic of the acetabulum and bone properties.

The characteristic of the acetabulum comprises its position, dimension and orientation. More specific, how the defected acetabulum positioned within the pelvis? What is the dimension in term of diameter, deepness, as well as the normal direction of the acetabulum and the thickness of the cartilage remained? How is the distribution of bone thickness around the acetabular shell in nature?

As will be appreciated by those of skill in the art, in practice of the present invention, all commercial available technologies in clinics are applicable for obtaining information listed above, such as, unless otherwise indicated, conventional methods of x-ray imaging and processing, x-ray tomosynthesis, ultrasound including A-scan, B-scan and C-scan, computed tomography (CT scan), magnetic resonance imaging (MRI), optical coherence tomography, single photon emission tomography (SPECT) and positron emission tomography (PET), as well as all software for analyzing data, within the skill of the art. Such techniques and their advantage/disadvantage are fully explained in the literatures and need not be described herein. In addition, the successful stories of applying such technologies for a purpose as same as obtaining patient's characteristic herein have reported in literatures as well. A detailed description and discussion of all possible protocol for information collection from patient is not included herein to avoid obscuring the invention, but would be apparent to those of skill in the art.

As will be also appreciated by those of skill in the art, in practice of the present invention, a diagnosis of bone property from each individual patient needed to be fulfilled by all available corresponding technologies for determining bone quality in order that surgeon estimates the elasticity of the bone with a respect of how well it can deal with press fitting method.

According to the information and analysis results above, the surgeon should be able to propose a customized protocol of surgery, for example, select a proper surgical kit(s) and a correct procedure affiliated for hip joint replacement of each individual patient prior to surgery. More particularly, parameters herein should at least include or be defined:

1) the dimension of the acetabular prosthesis intended to be used;

2) the dimension of the corresponding finishing reamer(s) intended to use and;

3) the orientation of the acetabulum with a respect to the pelvis in order to navigate operation and ensure proper attitude of the acetabular reamer.

2. A procedure of a primary, coarsely reaming the acetabulum 220: According to the procedure chosen prior to the surgery, a primary, coarsely reaming the acetabulum is conducted by following the pre-treatment mentioned above. A task of the primary reaming herein is to form a preliminary profile of the acetabular cavity with a clean, clear bone surface and a proper size. To be more specific, a criteria of such a cavity reamed herein includes sweeping all cartilage and a minimum layer of cancellous bone from the original acetabular surface, particular in apex area of cavity, until showing a few bloods “bleching out” from the all cancellous bone surface and that the cavity has been expanded up to a proper size, which is closer to intended dimension. To be more specific, the final size reamed by the primary reaming step 220 should be at least about 2 mm less than the dimension of acetabular cup selected.

3. A procedure of finishing, finely reaming the acetabulum 230: It is a customer reaming step that follows the primary reaming step. The customized reaming herein means that the size of the finishing reamer selected must base upon individual patient's information measured. The finishing reamer with a predefined, precise dimension has been used to finely correct any bias of the cavity had. No matter what kind of the cavity had from the coarse reaming, the finishing reamer 54 is able to remedy acetabulum up to an accurate dimension, which satisfies all requirements for precisely press-fitting the selected acetabular cup. Here there are two features being achieved by the finishing reamer to ensure the surgical quality: 1) To provide fresh cutting teeth. 2) To choose a right dimension of reamer by surgeon.

4. A procedure of installing the acetabular prosthesis on the site 240: surgeon fulfills the acetablar prosthesis into acetabulum reamed by following a standard procedure of implantation as usual.

A fourth embodiment regarding to a protocol 1:

With further reference to FIG. 8, a block diagram is showing a content of a preferred protocol 1 for installing an acetabular prosthesis cup into acetabulum reamed during a hip joint replacement surgery. Particularly, the protocol 1 is merely corresponding to the surgical kit, type 1, cited in the first preferred embodiment of surgical kit(s) (FIG. 3) above. As described above, the surgical kit(s)/package selected by surgeon according to the diagnosis from information of individual patient include an acetabular prosthesis assembly and one low cost, finishing reamer 54. Correspondingly, the preferred protocol 1 comprises the procedures/steps shown as following in processing sequence:

1. A procedure of information collection from patient and decision making 210: a detail of procedure is same as the step in the general protocol described above.

2. A procedure of coarsely reaming the acetabulum 222: the process could be still carried out, for example, by a set of conventional dome-like reamers as usual in order to create a preliminary profile of the cavity with the criteria mentioned in the general protocol above. As one in the regular processing step of a conventional reaming, the dome-like reamer head has been exchanged step by step until reaching the expecting size of the cavity.

3. A procedure of a finishing reaming 232: the fine reaming process of the acetabulum is implemented by the low cost, finishing reamer 54 herein packaged with the acetabular prosthesis intended to be used. The selection of size of the finishing reamer is based upon individual patient's information measured. The finishing reamer with a predefined size would accurately trim the cavity coarsely reamed in order to have a cavity with a predefined dimension and fine quality of the surface.

4. A procedure of the implantation of the acetabular prosthesis 240. The surgeon follows the standard procedure of the acetabular prosthesis as usual.

It is worth to emphasize that, through this protocol; one might realize that the patient information obtained herein might not be so critical or necessary on deciding the intended size of the acetabular prosthesis as well as the finishing reamer at the time prior to implantation. For example, in a case, if somehow surgeon or hospital is unable to implement the primary measurement of the patient listed above before surgery or they are uncertain on using such the results obtained, as a safe mode, surgeon could still decide what are the type and size of the surgical kit(s)/package intended to use at the time of him finishing the coarse reaming step. For example, after coarsely reaming step, the surgeon can make such a decision based upon both the size of the last reamer (the dome reamer) used and an intuitive feeling when he reamed the bone of patient, similar as one did in a convenient reaming procedure. If it is available, information of bone quality measured from the patient is also useful as a reference. For instance, after coarse reaming step, surgeon determines that a size of acetabular prosthesis intended to use is 52 mm according to the size (50 mm) of the last conventional reamer used. Simultaneously, he might also select a size or type of the finishing reamer by comprehensively considering the bone property measured from individual patient and the cutting feeling he experienced in his coarsely reaming. For example, 51.0 mm of reamer would be the best choice, if patient's bone quality is in a good shape. In operation room there will be a few type of packages available for 52 mm prosthesis with options of different size of the finishing reamer. So the type of the surgical kit elected could be 52B or 52-51.0.

It is interesting, according to this protocol herein, the size and spherical geometry of acetabulum reamed by dome-like reamers or others herein is not critical anymore in this coarsely reaming step, so the drawbacks from the dome-like reamer and its operation become less important and could be ignored or tolerated. To be an advantage of using dome-like reamer herein is that most of debris generated by coarsely reaming will be picked within the shell of the dome-like reamer as usual, if wanted. So within the finely reaming step, the finishing reamer 54 only needs to sweep out few staff of bone. It might be unnecessary to pick the debris up at all.

As will be also appreciated by those of skill in the art, the any conventional reamer set used in the prior art is still good option for the primary reaming step used in this protocol, so surgeon has a chance to be familiar with and getting used to the surgical kit and its protocol, as well as a single use reamer or likes. As a suitable product model for a primary penetration into the market, such a design of the surgical kit, type 1 and its affiliated protocol 1 herein might be an appropriate, safe option in the case, in which a surgeon might be not comfortable with a complete new technique yet. Indeed, such a combination of reamers used herein could be an intermediate or transition step toward a surgical protocol affiliated with a fully disposable or a low cost tooling or likes.

A fifth embodiment regarding to a protocol 2:

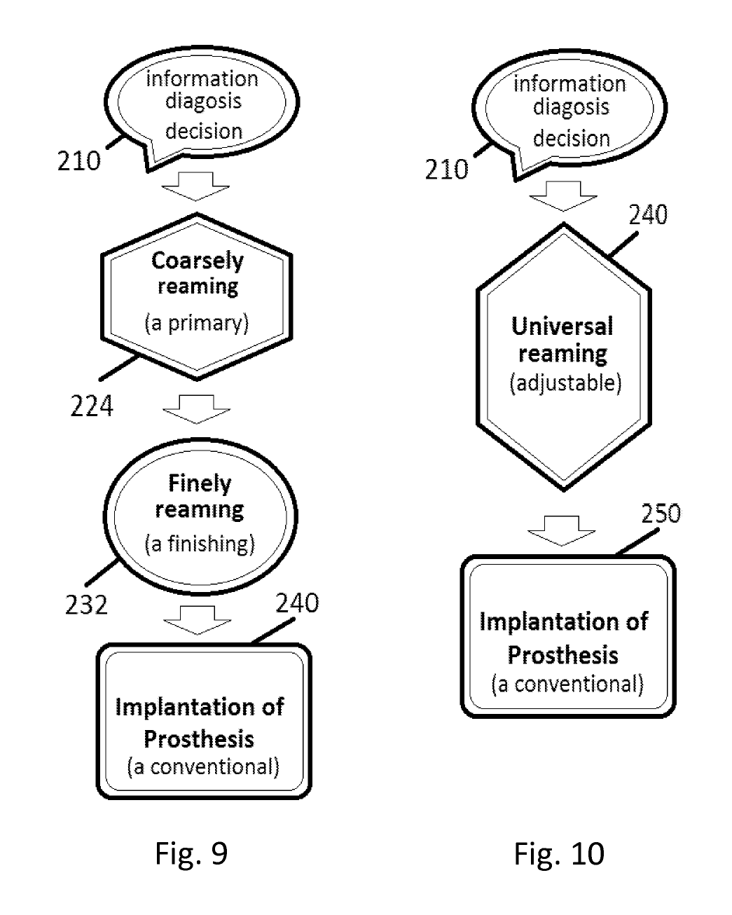

With further reference to FIG. 9, a block diagram of a preferred protocol 2 for installing an acetabular prosthesis cup during a hip joint replacement surgery is corresponding to the surgical kit, type 2, (also see FIG. 4) cited in the second preferred embodiment of surgical kit 44 above. The surgical kit 44 used herein is a specific package selected by surgeon according to the diagnosis from information of individual patient. As described above, the package selected by surgeon includes an acetabular prosthesis assembly 31 and a tooling set 50 that further comprises at least one low cost, primary reamer 52 and one low cost, finishing reamer 54. The preferred protocol 2 herein comprises the procedures/steps shown as followings in the processing sequence:

1. A procedure of information collection from patient and decision making 210: a detail of procedure is same as the same step in the general protocol above.

2. A procedure of coarsely reaming 224: In the contrary to the first preferred embodiment of the protocol 1, as described in FIG. 8, a coarsely reaming the acetabulum herein 224 herein is executed by a low cost, primary reamer 52 (in FIG. 4) packaged with the acetabular prosthesis together to create a preliminary profile of the cavity met with the criteria described above. Away from one in the regular processing step of a conventional reaming, the primary reamer 52 has a well-designed structure and fresh, sharp cutting elements and is able to fulfill a process of reaming from initial stage up to the expecting size of the acetabulum within a single step. In other word, one piece of the primary reamer replaces all work done by a set of dome-like conventional reamer in the coarse reaming step described in the preferred protocol 1 above.

3. A procedure of a fine reaming 232: its function and operation remain same as one described in the protocol 1 after the coarse reaming step (see FIG. 8). The finely, customized reaming processing is still implemented by the low cost, finishing reamer 54 (in FIG. 4) packaged together with the acetabular prosthesis selected.

4. A procedure of the implantation 240: The surgeon follows the standard procedure of the acetabular prosthesis as usual.

Since the size and spherical geometry of the cavity reamed by the coarsely reaming step is also not critical to the final results, in this protocol, it is expectable that the single primary reamer is able to at least replace full functions of the conventional dome-like reamer set done in the protocol 1. Furthermore, the combination of a fully or partial disposable primary and finishing reamer can fulfill the goal in more efficient and convenient manner and provide more advantages, such as safely and a significant a time and cost saving of the surgery.

A sixth embodiment regarding to a protocol 3:

With further reference to FIG. 10, a block diagram of a preferred protocol 3 herein for installing an acetabular prosthesis cup during a hip joint replacement surgery is corresponding to the surgical kit, type 3, (also see FIG. 6) as discussed in the third preferred embodiment of surgical kit 46 above. The surgical kit used herein is a specific package selected by surgeon according to the diagnosis based upon information of individual patient. As described above, the package selected includes, in briefly, an acetabular prosthesis assembly and one low cost, adjustable reamer 56, which can expand its reaming profile up to predefined dimension during the operation. In the contrary to the second embodiment of the preferred protocol 2 described in FIG. 9, the preferred protocol 3 herein combines the function of the coarsely and the finely reaming procedures in the previous embodiments together into one reaming step, that is fulfilled by one low cost, adjustable reamer 56. It is still a customized reaming, because the final size of the adjustable reamer used is still correlated with patient's bone information measured. The preferred protocol 3 herein comprises the procedures/steps shown as followings in processing sequence:

1. A procedure of information collection from patient and the decision making 210: a detail of the procedure is same as the step described in the general protocol.

2. A procedure of a reaming the acetabulum 250: a process of a primary reaming 220 and finishing reaming 230 the acetabulum described in the general procedure above has been merged into one step of reaming within this protocol. Such the reaming step has been carried out by a low cost, adjustable reamer 56 that has individually selected and packaged together with the acetabular prosthesis selected, in order to create a profile of the cavity with the criteria above. Away from one in the regular processing step of a conventional reaming and one within previous preferred embodiments above, the adjustable reamer head herein has a well-designed structure and a fresh cutting element and is able to fulfill a work from initial stage up to the expecting, final size of the cavity targeted within one step.

3. A procedure of the implantation 240: The surgeon follows the standard procedure of the acetabular prosthesis as usual.

Even though the reaming procedure herein has merged both primary and finishing reaming steps together, for a preferred example, a typical reaming step herein has still included both a primary and a final reaming stage, but respectively fulfilled by a single reamer. In other word, the reamer starts to ream the original acetabulum by its initial cutting profile in order to take mass of bone out from its surface, then progressively sizes up its cutting profile until reaching its high end of the size during the rotation, then the reamer would precisely ream the cavity up to the finishing condition of acetabulum.

Obviously, the preferred protocol 3 herein has shown more advantages in many aspects than others do. Such as it could be disposable, more convenient and run at a low cost. The further simplified procedure on reaming should be more significant in clinic beyond two previous protocols above. It is worth to mention, if this protocol described herein combines with a surgical robot (CAOS), the surgery of hip joint replacement should become more precise and efficient.

The above description is presented to enable any person skilled in the art to make and use the invention. Various modifications to the embodiments described will be readily apparent to those skilled in the art, and the generic principles defined herein can be applied to other.

What we claim:

1. A ready-for-use package of a sterilized surgical kit for installation of artificial acetabular prosthesis in hip replacement surgery comprising: a prosthesis compartment for packaging a set of acetabular prosthesis having a specific size; and a tooling compartment comprising at least a rotatable reamer for surgeon being able to select a cutting profile thereof and patient-specifically ream a defected acetabular site.

2. The package of the claim 1, wherein the tooling compartment further comprising one finishing reamer having a spherical cutting diameter selected from the group consisting of 0.5, 1.0, 1.5 or 2.0 mm smaller than the size of the corresponding prosthesis within the package.

3. The package of the claim 1, wherein the tooling compartment further comprising one primary reamer having a spherical cutting diameter being at least 3 mm smaller than the size of the prosthesis set included within the package and one finishing reamer having a spherical cutting diameter selected from the group consisting of 0.5, 1.0, 1.5 or 2.0 mm smaller than the size of the corresponding prosthesis within the package.

4. The package of the claim 1, wherein the tooling compartment comprising one size-expandable/adjustable reamer having a predefined and variable cutting range of the spherical cutting diameter, the rang corresponding to the size of the corresponding prosthesis included within the package.

5. The package of the claims 1 to 4, wherein all reamer(s) packaged within the tooling compartment are of any type of a single use or a low cost reusable, rotatable acetabular reamer, regardless a cutting element used, a cutting principle followed and a structure designed thereof.

6. The package of the claim 1, wherein the sterilized surgical kit has properly been cleaned, wrapped, sterilized and packaged in a standard manner.

7. A method for patient-specifically preparing acetabular cavity being in a full continuous hemispherical form and having a personalized dimension for hip replacement surgery comprising:
a step A of measuring and characterizing a defected acetabular site of the respective patient by standard methods prior to surgery for predetermining a size of acetabular prosthesis intended to be installed and a personalized orientation and dimension of a surgical site intended to be prepared; a step B of surgeon selecting the package according to the claims 1 to 4 for corresponding to a characterization of the surgical site from the step A; a step C of coarsely reaming the defected acetabular site for forming a desired profile having a depth matching the acetabular prosthesis and a preliminary dimension being at least 3 mm smaller than the acetabular prosthesis; and a step D of continuously reaming the site by rotating the finishing reamer selected until reaching a full hemispherical surface having the personalized dimension, which is able to tightly hold the prosthesis.

8. The method of claim 7, wherein the finishing reamer has a hemispherical cutting diameter respectively selected from 0.5, 1.0, 1.5 or 2.0 mm smaller than the size of the corresponding acetabular prosthesis co-packaged according to claims 1, 2 and 3.

9. The method of claim 7, wherein the step C comprises using a set of conventional dome-like acetabular reamer(s) to ream the site for forming the desired profile of the site.

10. The method of claims 7 and 8, wherein the step C comprises using the primary reamer to ream the site for forming the desired profile of the site and the step D comprises using the finishing reamer to further ream the site following the step C for patient-specifically reaming the defected acetabular site, respectively; the primary and finishing reamer packaged within the package according to the claim 3 and selected by surgeon.

11. The method of claim 7, wherein the step C and D are accomplished by the adjustable reamer in a successive manner of continuously actuating the cutting range of the adjustable reamer until reaching an expected cutting profile thereof for creating the full hemispherical surface having the personalized dimension determined in the step A; the adjustable reamer packaged within the package according to the claim 4 selected by surgeon.
